(19) United States
(12) Patent Application Publication
(10) Pub. No.: US 2009/0012361 A1
(43) Pub. Date: Jan. 8, 2009

(54) Apparatus and methods relating to color imaging endoscope systems

(75) Inventors: Nicholas B. MacKinnon, Vancouver (CA); Ulrich Stange, Vancouver (CA)

Correspondence Address: GRAYBEAL JACKSON LLP, 155 - 108TH AVENUE NE, SUITE 350, BELLEVUE, WA, 98004-5973, US

(73) Assignee: Tidal Photonics, Inc., Vancouver (CA)

(21) Appl. No.: 12/072,493

(22) Filed: Feb. 25, 2008

Related U.S. Application Data
(63) Continuation of application No. 10/951,439, filed on Sep. 27, 2004, now abandoned.
(60) Provisional application No. 60/506,264, filed on Sep. 26, 2003.

(51) Int. Cl. A61B 1/045

(52) U.S. Cl. 600/118

(57) ABSTRACT

Color endoscopes, light sources and endoscopy systems, etc., that have good dynamic range and/or resolution while reducing the size and cost of the endoscopes. The endoscopes achieve this, in part, by using a black and white (grayscale or monochromatic) sensor at the tip of the endoscope instead of a color sensor. The endoscope uses a light system that precisely and specifically illuminates the tissue one color at time, captures the image in grayscale, then uses a computer to associate the image with the color. Certain aspects of the invention apply to imaging systems in addition to endoscopes.

### CROSS-REFERENCE TO RELATED APPLICATIONS

The present application claims priority from pending U.S. provisional patent application No. 60/506,264 filed Sep. 26, 2003.

### BACKGROUND

The diagnosis and treatment of disease often requires a device to view the interior passages of the body or body cavities that may have to be accessed by surgical instruments. The most common way to do this is via endoscopy systems. Endoscopes are well known as devices to relay images of the internal anatomy to the eye of a physician or surgeon. They include flexible endoscopes such as bronchoscopes, gastroscopes, colonoscopes, sigmoidoscopes and others. They also include rigid endoscopes such as arthroscopes, laparoscopes, cystoscopes, uretoscopes and others. Endoscopes may use optical, fiberoptic or electronic devices or systems to relay images to the operator. Endoscopes are typically part of an imaging system. The imaging system usually comprises light sources, cameras, image recording devices and image display devices such as video monitors or printers.

Endoscopes have become smaller and less expensive to build and have resulted in a continuing improvement in image quality. Newer and smaller imaging sensors such as charge coupled devices (CCDs) or complementary metal oxide semiconductor (CMOS) image sensors have allowed the cameras to record and transmit a video image to be integrated into the tip of the endoscope.

A problem with integrating these image sensors into the small space available at the tip of an endoscope is that compromises in either image resolution or image dynamic range are usually required. Resolution is the ability to spatially resolve details in an image. Dynamic range refers to range of shades of light and dark that can be captured by the imaging device. A limiting factor for resolution is usually not the optical quality of the endoscope lenses but the number of pixels available on the CCD. A limiting factor for dynamic range is the ability of each pixel of the CCD to capture the light that makes up an image. Smaller image sensors require smaller pixels, and smaller pixels mean less ability to capture a wide range of light levels.

Most endoscopes are equipped with image sensors that can capture a color image when the tissue is illuminated by white light. This is usually accomplished by placing optical filters that transmit different colors over adjacent pixels on the image sensor. Usually these filters are red, green and blue filters, but they may also be other colors such as cyan, yellow and magenta, or other combinations of colors as may be desired. These filters are commonly arranged in a repeating spatial pattern wherein filters of different colors are located over pixels adjacent to one another. A common pattern of red, green and blue pixels is a Bayer pattern. The adjacent color filtered pixels are each assigned the same spatial location in the digital image, even though they are not actually in the same location and thus the features of the image they are measuring are not in the identical spatial location. Usually these pixels are close enough to approximate the optical characteristics of the tissue being imaged, but they may in some cases reduce the ability to accurately locate details, such as networks of blood vessels. In contrast, when the detector's pixels are actually measuring the same location in the image the measurement can be more accurate.

One method of improving the accuracy of imaging can be to use three image sensors maintained at the proximal end of the endoscope. Such sensors split the image into three wavelength components, each with its own image path, so that the images are registered accurately on each image sensor. These types of image sensors are commonly called 3-CCD cameras and are commercially available from companies such as Sony Corporation of Japan. These devices are feasible and produce high quality images when the endoscope relays an optical image outside of the body cavity, rather than transmitting an electronic image, but are costly and cannot be easily implemented in the tip of an endoscope.

Another method of producing high quality images is to use a single monochrome CCD and to sequentially capture images illuminated by different wavelengths of illumination light by changing a filter in front of the sample or target. Such systems have been produced using optical filter wheels as with an endoscope system produced by Pentax Corporation of Japan and have also been produced using liquid crystal color filters or acousto-optic tunable filters placed in front of cameras, such as those available from QImaging Corporation of Vancouver, Canada. While the liquid crystal and acousto-optic filters have good control of exposure time, none are currently available placed at the tip of an endoscope.

Endoscopes with monochrome CCDs have been produced and used in conjunction with rotating filter wheels by Pentax Corporation but these have the disadvantage of fixed exposure duration and fixed relative brightness provided by the filters in the rotating filter wheel.

Thus, there has gone unmet a need for endoscopes and endoscopy systems that can provide smaller and lower cost endoscopes while maintaining or improving image qualities such as resolution and dynamic range. The present invention provides these and other advantages.

### SUMMARY

The present invention provides endoscopes, light sources and endoscopy systems, etc., that have good dynamic range and/or resolution while reducing the size and cost of endoscopes. The imaging systems of the present invention can also apply to any imaging system wherein it may be desirable to utilize monochromatic sensors with computer-controlled illumination light systems such that the system can provide substantially true-color images of an object or scene or other target. Such systems can include video cameras, digital cameras, mini-imaging systems such as mini-cameras, industrial imaging systems for Q/A or manufacturing or otherwise as desired.

In some embodiments, the endoscopy systems comprise an endoscope with an integrated image sensor or video camera at the distal tip or portion of the endoscope. Generally speaking, the distal end of an endoscope is the end of the endoscope that is inserted into the body and directed to a target tissue; the proximal end is the end of the endoscope that is maintained outside the body, and typically comprises an ocular eyepiece and one or more handles, knobs and/or other control devices that allow the user to manipulate the distal end of the endoscope or devices located at the distal end of the endoscope. As used herein, the distal end of the endoscope includes the distal tip of the endoscope, which is the most distal surface or opening of the endoscope, and the portion of the endoscope adjacent to the distal tip of the endoscope. Endoscopes generally are well known. U.S. Pat. No. 6,110,106; U.S. Pat. No. 5,409,000; U.S. Pat. No. 5,409,009; U.S. Pat. No. 5,259,837; U.S. Pat. No. 4,955,385; U.S. Pat. No. 4,706,681; U.S. Pat. No. 4,582,061; U.S. Pat. No. 4,407,294; U.S. Pat. No. 4,401,124; U.S. Pat. No. 4,204,528; U.S. Pat. No. 5,432,543; U.S. Pat. No. 4,175,545; U.S. Pat. No. 4,885,634; U.S. Pat. No. 5,474,519; U.S. Pat. No. 5,092,331; U.S. Pat. No. 4,858,001; U.S. Pat. No. 4,782,386; U.S. Pat. No. 5,440,388.

The endoscope or other imaging system can further comprise an illumination light guide, typically an optical fiber, fiber bundle, lens or combination of these or other optical relay systems, that transmits light from a light source and projects it to illuminate the anatomical site or other target being imaged.

The video camera can be an imaging sensor such as a CMOS or CCD image sensor and an objective lens that forms an image of the anatomical site on the image sensor. The image sensor is a monochrome image sensor without a matrix of color filters superimposed on the sensor.

The image sensor can be operated under computer control and can be synchronized with a computer controlled light source.

The computer controlled light source can comprise a lighting system that comprises at least one bright source of broad-band visible illumination commonly called white light, a wavelength dispersive element such as a prism or diffraction grating and a reflective pixelated spatial light modulator (RPSLM) such as a digital micromirror device or liquid crystal on silicon (LCOS), or other suitable tunable light filter such as a transmissive pixelated spatial light modulator, or acousto-optic tunable filter (AOTF). The light from the light source is directed as a beam to the wavelength dispersive element which disperses the beam into a spectrum that is imaged onto a RPSLM. The pixel elements of the RPSLM can be switched to select wavelengths of light and selected amounts of the selected wavelengths of light to propagate. The light source can also comprise a plurality of different light emanators, for example to provide greater total intensity or each providing a different wavelength or wavelength band(s) of light in combination with a selective device(s) configured to transmit desired amounts of the different wavelength band(s). Exemplary light sources include red, green and blue LEDs or other desired lamps and photon generators, and exemplary selective devices include rheostats that control the power and thus output of the light sources, as well as various other wavelength and intensity selective elements discussed herein. The light that propagates is then, if desired, optically mixed together and directed to the illumination path of an endoscope or other medical device.

The RPSLM may be operably connected to a controller, which controller contains computer-implemented programming that controls the on/off pattern of the pixels in the RPSLM. The controller can be located in any desired location to the rest of the system. For example, the controller can be either within a housing of the source of illumination or it can be located remotely, connected by a wire, cellular link or radio link to the rest of the system. If desired, the controller, which is typically a single computer but can be a plurality of linked computers, a plurality of unlinked computers, computer chips separate from a full computer or other suitable controller devices, can also contain one or more computer-implemented programs that provide specific lighting characteristics, i.e., specific desired, selected spectral outputs and wavelength dependent intensities, corresponding to known wavelength bands that are suitable for or a specific light for disease diagnosis or treatment, or to invoke disease treatment (for example by activating a drug injected into a tumor in an inactive form), or other particular situations.

In one aspect, a lighting system provides a variable selected spectral output and a variable wavelength dependent intensity distribution. The lighting system comprises a light path that comprises: a) a spectrum former configured to provide a spectrum from a light beam traveling along the light path, and b) a reflective pixelated spatial light modulator (RPSLM) or other rapid, finely controlled wavelength tunable filter, located downstream from and optically connected to the spectrum former, the RPSLM reflecting substantially all light impinging on the RPSLM and switchable to reflect light from the light beam between at least first and second reflected light paths, at least one of which does not reflect back to the spectrum former. The RPSLM can be a digital micromirror device. The RPSLM is operably connected to at least one controller containing computer-implemented programming that controls an on/off pattern of pixels in the RPSLM to reflect a desired segment of light in the spectrum to the first reflected light path and reflect substantially all other light in the spectrum impinging on the RPSLM to at least one of the second reflected light path and another reflected light path that typically does not reflect back to the spectrum former, the desired segment of light consists essentially of a desired selected spectral output and a desired wavelength dependent intensity distribution.

In some embodiments, the system further comprises a light source located upstream from the spectrum former, and the spectrum former comprises at least one of a prism and a diffraction grating, which can be a reflective diffraction grating, transmission diffraction grating, variable wavelength optical filter, or a mosaic optical filter. The system may or may not comprise, between the spectrum former and the RPSLM, an enhancing optical element that provides a substantially enhanced image of the spectrum from the spectrum former to the RPSLM. The RPSLM can be a first RPSLM, and the desired segment of light can be directed to a second RPSLM operably connected to the same controller or another controller containing computer-implemented programming that controls an on/off pattern of pixels in the second RPSLM to reflect the desired segment or other segment of light in one direction and reflect other light in the spectrum in at least one other direction. The system can further comprise an optical projection device located downstream from the first RPSLM to project light out of the lighting system as a directed light beam.

The desired segment of light can, for example, be selected to substantially mimic a spectral output and a wavelength dependent intensity distribution of at least one of the output energy for disease treatment, photodynamic therapy, or disease diagnosis, or to enhance contrast for detection or discrimination of a desired object in a scene.

In some embodiments, the systems comprise an optical concentrator that concentrates light from the light source. For example, the concentrator can comprise light from an arc lamp or other point source is directed as a beam through an aperture stop. The aperture stop blocks out of focus light to prevent it from propagating through the system and degrading optical performance. In focus light is collected by a collimating lens and the collimated light is directed to a cylindrical lens. Collimated light is light in which the directions of propagation of the rays of light making up the beam are substantially parallel. The cylindrical lens focuses the light in only the horizontal axis resulting in convergence of the collimated beam into a line of light with a mean angle of incidence at focal plane. The reflective microarray is positioned at the focal plane and oriented to reflect the beam incident on its surfaces while rotating the angles of convergence or divergence of portions of the line of light through 90 degrees. The size of the portion so rotated is determined by the spatial period of the reflective microarray.

In another aspect, a stand alone light source is sized to project light onto a tissue and having a variable selected spectral output and wavelength dependent intensity distribution. The source of illumination can comprise a) a high output light source, b) a spectrum former optically connected to and downstream from the light source to provide a spectrum from a light beam emitted from the light source, c) an enhancing optical element optically connected to and downstream from the spectrum former that provides an enhanced image of the spectrum; d) a RPSLM located downstream from and optically connected to the spectrum former, the RPSLM reflecting substantially all light impinging on the RPSLM and switchable between at least first and second reflected light paths, wherein the RPSLM can be operably connected to at least one controller containing computer-implemented programming that controls an on/off pattern of pixels in the RPSLM to reflect a desired segment of light in the spectrum in first reflected light path and reflect other light in the spectrum to at least one of the second reflected light path and another reflected light path that does not reflect back to the spectrum former, the desired segment of light consisting essentially of a desired selected spectral output and a desired wavelength dependent intensity distribution; and, e) a projection system optically connected to and downstream from the RPSLM in the first direction, wherein the projection system projects the desired segment as a directed light beam to illuminate the tissue. Similar systems can also be provided wherein the systems comprise multiple differential light sources coupled with selective filters, and/or with RPSLMs comprising liquid crystal on silicon (LCOS), e.g., LCOS systems made by Intel and discussed at http://www.intel.com/design/celect/technology/lcos/, MEMS, or other technology that can be configured to provide the wavelength selective and intensity selective illumination light discussed herein, or other desired light combinations suitable for use with the sensors, processors, etc., of the endoscopes discussed herein.

The source of illumination can further comprise a detector optically connected to and downstream from the RPSLM, the detector also operably connected to a controller containing computer-implemented programming configured to determine from the detector whether the desired segment contains a desired selected spectral output and a desired wavelength dependent intensity distribution, and adjust the on/off pattern of pixels in the RPSLM to improve the correspondence between the desired segment and the desired selected spectral output and the desired wavelength dependent intensity distribution. The source of illumination can also comprise a heat removal element operably connected to the light source to remove undesired energy emitted from the light source toward at least one of the RPSLM, the enhancing optical element, and the spectrum former.

The various aspects, embodiments, elements, etc., discussed herein can be combined and permuted as desired. For example, the sources of illumination and lighting systems, as well as methods, kits, and the like related to them, etc., can comprise various elements discussed for each other even if the elements are specifically discussed only for the other (for example, the detector of the source of illuminations can also be suitable for use with the lighting system).

The heat removal element can be located between the spectrum former and the first reflective spatial light modulator, between the lamp and the spectrum former, or elsewhere as desired. The heat removal element can comprise a dichroic mirror. The dichroic mirror transmits desired wavelengths and reflects undesired wavelengths, or vice-versa. The undesired energy can be directed to an energy absorbing surface and thermally conducted to a radiator. The heat removal element can be an optical cell containing a liquid that absorbs undesired wavelengths and transmits desired wavelengths. The liquid can be substantially water and can flow through the optical cell via an inlet port and outlet port in a recirculating path between the optical cell and a reservoir. The recirculating path and the reservoir can comprise a cooling device, which can be a refrigeration unit, a thermoelectric cooler, or a heat exchanger.

The source of illumination further can comprise a spectral recombiner optically connected to and located downstream from the pixelated spatial light modulator, which can comprise a prism, a Lambertian optical diffusing element, a directional light diffuser such as a holographic optical diffusing element, a lenslet array, or a rectangular light pipe. In one embodiment, the spectral recombiner can comprise an operable combination of a light pipe and at least one of a lenslet array and a holographic optical diffusing element. A detector can be located in the at least one other direction, and can comprise at least one of a CCD, a CID, a CMOS, and a photodiode array. The high output light source, the spectrum former, the enhancing optical element that provides an enhanced image, the RPSLM, and the projection system, can all be located in a single housing, or fewer or more elements can be located in a single housing.

In another aspect the light source or endoscopy system comprises an adapter or other apparatus for mechanically and/or optically connecting the illumination light guide of an endoscope to the output of the light source. The illumination light guide of the endoscope can be at least one of an optical fiber, optical fiber bundle, liquid light guide, hollow reflective light guide, or free-space optical connector or other light guide as desired. The light guide may be integral with the remainder of the endoscope or it may be modular and separable from the endoscope.

In another aspect the endoscope comprises a longitudinal tube of a biologically compatible and suitable material such as stainless steel or a suitable polymer that may be inserted into the body and that is equipped with an objective lens, and an image sensor and a light output port at the distal tip of the endoscope, typically sealed or encapsulated for cleaning or sterilization. The objective lens and/or the illumination path may comprise a beam steering mirror or prism or other beam director for side or angle viewing of a tissue. The endoscope may further provide a lumen that provides for insertion of a tissue sampling accessory such as a brush or biopsy forceps, or a treatment accessory such as an electrosurgical loop or optical fiber or other accessory.

In some embodiments the image sensor of the endoscope can be an unfiltered image sensor. An unfiltered image sensor relies on the natural optical response of the sensor material to light impinging on the sensor to generate an image signal.

In other embodiments the image sensor can have an optical filter placed in front of it to limit the wavelengths of light that reach the sensor. Unlike a matrix filter that only allows selected wavelengths to reach selected pixels, the optical filter is configured to allow the same wavelengths to reach all pixels if they are present in the signal from the sample. The optical filter can be at least one of a long-pass filter, a short-pass filter, a band-pass filter, or a band-blocking filter. A long-pass filter is useful to block undesired wavelengths such as ultraviolet light or fluorescence excitation light from impinging on the sensor. A short-pass filter is useful to block undesired wavelengths such as infrared light from impinging on the sensor. A band-pass filter may be useful to allow only selected wavelengths such as visible light to impinge on the detector. A band-blocking filter is useful to block fluorescence excitation light from impinging on the image sensor.

In some embodiments, the computer controlled image sensor (CCIS) can be synchronized to the computer controlled light source (CCLS) to provide sequences of images of tissue illuminated by desired wavelengths of light and captured as digital images. These digital images can then be combined or processed as desired to provide useful information to the physician or surgeon.

The CCLS and CCIS are operably connected to a controller, which controller contains computer-implemented programming that controls the time of image acquisition in the CCIS and the wavelength distribution and duration of illumination in the CCLS. The controller can be located in any desired location relative to the rest of the system. For example, the controller can be either within a housing of the source of illumination or it can be located remotely, connected by a wire, cellular link or radio link to the rest of the system. If desired, the controller, which is typically a single computer but can be a plurality of linked computers, a plurality of unlinked computers, computer chips separate from a full computer or other suitable controller devices, can also contain one or more computer-implemented programs that provide control of image acquisition and/or control of specific lighting characteristics, i.e., specific desired, selected spectral outputs and wavelength dependent intensities, corresponding to known wavelength bands that are suitable for imaging or a specific light for disease diagnosis or treatment, or to invoke disease treatment (for example by activating a drug injected into a tumor in an inactive form), or other particular situations.

The endoscopy system can further comprise computer controlled image acquisition and processing systems that can analyze the information from an image or sequence of images and present it in a way that is meaningful to an operator.

In a further aspect, the present methods comprise illuminating a tissue comprising: a) directing a light beam along a light path and through a spectrum former to provide a spectrum from the light beam traveling; b) reflecting the spectrum off a RPSLM that can be operably connected to at least one controller containing computer-implemented programming that controls an on/off pattern of pixels in the RPSLM, wherein the reflecting can comprise reflecting a desired segment of light in the spectrum in a first reflected light path that can be not back to the spectrum former and reflecting substantially all other light in the spectrum impinging on the RPSLM in at least one second reflected light path that can be not back to the spectrum former, to provide a modified light beam consisting essentially of a selected spectral output and a selected wavelength dependent intensity distribution. In some embodiments, the methods include illuminating tissue using other illumination systems within the scope of the concepts discussed herein.

The methods further can comprise emitting the light beam from a light source located in a same housing as and upstream from the spectrum former. The methods further can comprise switching the modified light beam between the first reflected light path and the second reflected light path. The methods further can comprise passing the light beam by an enhancing optical element between the spectrum former and the RPSLM to provide a substantially enhanced image of the spectrum from the spectrum former to the reflective pixelated spatial light modulator. The reflective pixelated spatial light modulator can be a first reflective pixelated spatial light modulator, and the methods further can comprise reflecting the modified light beam off a second reflective pixelated spatial light modulator operably connected to at least one controller containing computer-implemented programming that controls an on/off pattern of pixels in the second RPSLM to reflect the desired segment of light in one direction and reflect other light in the spectrum in at least one other direction.

The methods further can comprise passing the modified light beam by an optical projection device located downstream from at least one of the first RPSLM and the second RPSLM to project light as a directed light beam.

The methods of illuminating a tissue can also comprise: a) directing a light beam along a light path and through a spectrum former to provide a spectrum from the light beam traveling; and, b) passing the spectrum via a pixelated spatial light modulator located downstream from and optically connected to the spectrum former, the pixelated spatial light modulator operably connected to at least one controller containing computer-implemented programming that controls an on/off pattern of pixels in the pixelated spatial light modulator, wherein the on/off pattern can be set to pass a desired segment of light in the spectrum in one direction and interrupt other light in the spectrum impinging on the pixelated spatial light modulator, to provide a modified light beam consisting essentially of a selected spectral output and a selected wavelength dependent intensity distribution, and wherein the methods do not comprise passing the spectrum by an enhancing optical element between the spectrum former and the pixelated spatial light modulator that provides an enhanced image of the spectrum from the spectrum former to the pixelated spatial light modulator.

In still other aspects, the methods comprise emitting modified light consisting essentially of a desired selected spectral output and a desired wavelength dependent intensity distribution from an endoscopy light source. The methods can comprise: a) emitting light from a high output light source located in a housing of the luminaire; b) passing the light by a spectrum former optically connected to and downstream from the light source to provide a spectrum from a light beam emitted from the light source; c) passing the spectrum by an enhancing optical element connected to and downstream from the spectrum former to provide an enhanced image of the spectrum; d) reflecting the spectrum off a RPSLM that can be operably connected to at least one controller containing computer-implemented programming that controls an on/off pattern of pixels in the RPSLM, wherein the reflecting can comprise reflecting a desired segment of light in the spectrum in a first reflected light path that can be not back to the spectrum former and reflecting substantially all other light in the spectrum impinging on the RPSLM in at least one second reflected light path that can be not back to the spectrum former, to provide a modified light beam consisting essentially of a selected spectral output and a selected wavelength dependent intensity distribution; and, e) passing the modified light beam by a projection system optically connected to and downstream from the RPSLM in the first direction, wherein the projection system projects the modified light beam from the source of illuminations as a directed light beam.

The methods can further comprise reflecting the desired segment of light to a detector optically connected to and downstream from the RPSLM, the detector located in the second reflected light path or otherwise as desired and operably connected to the controller, wherein the controller contains computer-implemented programming configured to determine from the detector whether the desired segment contains the desired selected spectral output and the desired wavelength dependent intensity distribution, and therefrom determining whether the first segment contains the desired selected spectral output and the desired wavelength dependent intensity distribution. The methods can comprise adjusting the on/off pattern of pixels in the RPSLM to improve the correspondence between the desired segment and the desired selected spectral output and the desired wavelength dependent intensity distribution.

The methods can also comprise removing undesired energy emitted from the light source toward at least one of the RPSLM, the enhancing optical element, and the spectrum former, the removing effected via a heat removal element operably connected to the light source. The methods further can comprise a spectral recombiner optically connected to and located downstream from the RPSLM.

The methods can further comprise directing the output beam to illuminate a tissue by at least one of directly illuminating the tissue via a projected beam, or directing the beam into the light guide of an endoscope, or directing the beam into the light guide of a surgical microscope or other imaging system for viewing tissue or other objects, for example imaging systems suitable for use with machine vision.

The methods can further comprise capturing an image of the light emitted by a tissue illuminated by the light from the CCLS and storing it for processing, analysis or display.

The methods can further comprise combining a sequence of digital or analog images and processing or combining them to form an image of the tissue that provides information to the physician or surgeon.

The methods can comprise capturing and displaying a sequence of images where the wavelengths are substantially in the red, green and blue portions of the wavelength spectrum and the images are combined to produce a color image with the red green and blue channels.

These and other aspects, features and embodiments are set forth within this application, including the following Detailed Description and attached drawings. The discussion herein provides a variety of aspects, features, and embodiments; such multiple aspects, features and embodiments can be combined and permuted in any desired manner. In addition, various references are set forth herein, including in the Cross-Reference To Related Applications, and U.S. Pat. No. 6,781,691, and pending U.S. patent application Ser. No. 10/893,132, entitled Apparatus And Methods Relating To Concentration And Shaping Of Illumination, filed Jul. 16, 2004, that discuss certain apparatus, systems, methods, or other information; all such references are incorporated herein by reference in their entirety and for all their teachings and disclosures, regardless of where the references may appear in this application.

### BRIEF DESCRIPTION OF THE DRAWINGS

FIG. 1 provides a schematic depiction of a color endoscopy system comprising a carefully controlled light source and a gray scale sensor.

FIG. 2 provides a schematic depiction of how the computer controlled light source can modify the spectral distribution of the light that illuminates the tissue.

FIG. 3 provides a schematic depiction of a temporal sequence of three wavelength bands of illuminations produced by the CCLS and three resultant gray scale images captured by the CCIS.

FIG. 4 is a schematic representation of the assignment of the three monochrome images of FIG. 3 to the red, green and blue channels of a RGB color image and the resultant color image.

FIG. 5 is a schematic representation of how the CCLS can selectively sweep through a sequence of wavelength bands and capture an image at each stage of the sequence, which can then be assembled as a multispectral or hyperspectral image cube.

FIG. 6 is a schematic representation of how the CCLS can produce a spectral profile that can enhance or reduce contrast for a particular anatomical feature.

FIG. 7 is a schematic representation of a) a prior art sensor showing the rectangular arrangement of pixels on the image sensor, b) the arrangement of filters on a Bayer pattern image sensor and how the RGB filtered pixels are combined to create an effective pixel, and c) the relative size of the Bayer pattern image sensor from FIG. 7b and a monochrome image sensor with the same number of effective pixels.

FIG. 8 is a schematic representation of an endoscope tip illustrating the effect of image sensor size on endoscope diameter.

FIG. 9 is a flow chart depicting a power management scheme according to the present invention.

### DETAILED DESCRIPTION

The present invention provides color endoscopes, light sources and endoscopy systems, etc., that have good dynamic range and/or resolution while reducing the size and cost of endoscopes. The endoscopes achieve this, in part, by using a black and white (grayscale) sensor at the tip of the endoscope instead of a color sensor. The grayscale sensor is cheaper and smaller than the color sensor. The endoscope still provides a color image, however, by using a special light system that precisely and specifically illuminates the tissue in only a single color at time, captures the image in grayscale, then uses a computer to associate the image with the color. For example, red light is used to make an image, which is captured as a grayscale image, then reassigned as a red image after capture. The same is done with a blue image and green image, and then the three images are combined to provide a traditional red-green-blue (rgb) color image. The technique can also be done with other color combinations and numbers of colors. The present invention provides additional advantages as discussed further herein.

The lighting systems comprise a spectrum former upstream from an SLM such as an RPSLM, the SLM reflecting substantially all of the light in the spectrum into at least two different light paths, none of which reflect back to the light source or the spectrum former. At least one of the light paths acts as a projection light path and transmits desired light out of the lighting system. The lighting systems provide virtually any desired color(s) and intensity(s) of light, and avoid overheating problems by deflecting unwanted light and other electromagnetic radiation—and therefore unwanted heat—out of the system or to a heat management system. Thus, the heat is removed from the optical elements of the system. The systems can be part of another system, a luminaire, or any other suitable light source. The systems can provide virtually any desired light, from the light seen at the break of morning to specialized light for treating cancer or psoriasis, and may change color and intensity at speeds that are perceptually instantaneous, for example in less than a millisecond.

Turning to some general information about light, the energy distribution of light is what determines the nature of its interaction with an object, compound or organism. A common way to determine the energy distribution of light is to measure the amount or intensity of light at various wavelengths to determine the energy distribution or spectrum of the light. To make light from a light source useful for a particular purpose it can be conditioned to remove undesirable wavelengths or intensities, or to enhance the relative amount of desirable wavelengths or intensities of light.

A high signal to noise ratio and high out of band rejection enhances the simulation of the spectral characteristics of different light sources or lighting environments, and also enhances fluorescence excitation, spectroscopy or clinical treatments such as photodynamic therapy.

The systems and methods, including kits and the like comprising the systems or for making or implementing the systems or methods, provide the ability to selectively, and variably, decide which colors, or wavelengths, from a light source will be projected from the system, and how strong each of the wavelengths will be. The wavelengths can be a single wavelength, a single band of wavelengths, a group of wavelengths/wavelength bands, or all the wavelengths in a light beam. If the light comprises a group of wavelengths/wavelengths bands, the group can be either continuous or discontinuous. The wavelengths can be attenuated so that the relative level of one wavelength to another can be increased or decreased (e.g., decreasing the intensity of one wavelength among a group of wavelengths effectively increases the other wavelengths relative to the decreased wavelength). This is highly advantageous because such fine control of spectral output and wavelength dependant intensity distribution permits a single lighting system to provide highly specialized light such as light for diagnosing or treating disease or activating drugs, as well the ability to substantially mimic desirable lighting conditions such as a known lamp, a cathode ray tube image display device, a light emissive image display device, a desired natural ambient lighting scenario such as light at a specific longitude, latitude and weather condition, firelight, candlelight, or sunlight, or other sources of optical radiation.

### DEFINITIONS

The following paragraphs provide definitions of some of the terms used herein. All terms used herein, including those specifically discussed below in this section, are used in accordance with their ordinary meanings unless the context or definition indicates otherwise. Also unless indicated otherwise, except within the claims, the use of “or” includes “and” and vice-versa. Non-limiting terms are not to be construed as limiting unless expressly stated (for example, “including” and “comprising” mean “including without limitation” unless expressly stated otherwise).

A “controller” is a device that is capable of controlling a spatial light modulator, a detector or other elements of the apparatus and methods herein. A “controller” contains or is linked to computer-implemented programming. Typically, a controller comprises one or more computers or other devices comprising a central processing unit (CPU) and directs other devices to perform certain functions or actions, such as the on/off pattern of the pixels in the pixelated SLM, the on/off status of pixels of a pixelated light detector (such as a charge coupled device (CCD) or charge injection device (CID)), and/or compile data obtained from the detector, including using such data to make or reconstruct images or as feedback to control an upstream spatial light modulator. A computer comprises an electronic device that can store coded data and can be set or programmed to perform mathematical or logical operations at high speed. Controllers are well known and selection of a desirable controller for a particular aspect is readily achievable in view of the present disclosure.

A “spatial light modulator” (SLM) is a device that is configured to selectively modulate light. The present invention comprises one or more spatial light modulators disposed in the light path of an illumination system. A pixelated spatial light modulator comprises an array of individual pixels, which are a plurality of spots that have light passing characteristics such that they transmit, reflect or otherwise send light along a light path, or instead block the light and prevent it or interrupt it from continuing along the light path. Such pixelated arrays are well known in the art, having also been referred to as a multiple pattern aperture array, and can be formed by an array of ferroelectric liquid crystal devices, liquid crystal on silicon (LCOS) devices, electrophoretic displays, or by electrostatic microshutters. See, U.S. Pat. No. 5,587,832; U.S. Pat. No. 5,121,239; R. Vuelleumier, Novel Electromechanical Microshutter Display Device, Proc. Eurodisplay '84, Display Research Conference September 1984.

A reflective pixelated SLM_comprises an array of highly reflective mirrors that are switchable between at least an on and off state, for example between at least two different angles of reflection or between present and not-present. Examples of reflective pixelated SLMs include digital micromirror devices (DMDs), liquid crystal on silicon (LCOS) devices, as well as other MicroElectroMechanical Structures (MEMS). DMDs can be obtained from Texas Instruments, Inc., Dallas, Tex., U.S.A. In this embodiment, the mirrors have three states. In a parked or “0” state, the mirrors parallel the plane of the array, reflecting orthogonal light straight back from the array. In one energized state, or a “−10” state, the mirrors fix at −10° relative to the plane of the array. In a second energized state, or a “+10” state, the mirrors fix at +10° relative to the plane of the array. Other angles of displacement are possible and are available in different models of this device. When a mirror is in the “on” position light that strikes that mirror is directed into the projection light path. When the mirror is in the “off” position light is directed away from the projection light path. On and off can be selected to correspond to energized or non-energized states, or on and off can be selected to correspond to different energized states. If desired, the light directed away from the projection light path can also be collected and used for any desired purpose (in other words, the DMD can simultaneously or serially provide two or more useful light paths).

The pattern in the RPSLM can be configured to produce two or more spectral and intensity distributions simultaneously or serially, and different portions of certain RPSLMs can be used to project or image along two or more different projection light paths.

An “illumination light path” is the light path from a light source to a target, while a “detection light path” is the light path for light emanating from the target or sample to a detector. The light includes ultraviolet (UV) light, blue light, visible light, near-infrared (NIR) light and infrared (IR) light.

“Upstream” and “downstream” are used in their traditional sense wherein upstream indicates that a given device is closer to a light source, while downstream indicates that a given object is farther away from a light source.

The discussion herein includes both means plus function and step plus function concepts. However, the terms set forth in this application are not to be interpreted in the claims as indicating a “means plus function” relationship unless the word “means” is specifically recited in a claim, and are to be interpreted in the claims as indicating a “means plus function” relationship where the word “means” is specifically recited in a claim. Similarly, the terms set forth in this application are not to be interpreted in method or process claims as indicating a “step plus function” relationship unless the word “step” is specifically recited in the claims, and are to be interpreted in the claims as indicating a “step plus function” relationship where the word “step” is specifically recited in a claim.

Other terms and phrases in this application are defined in accordance with the above definitions, and in other portions of this application.

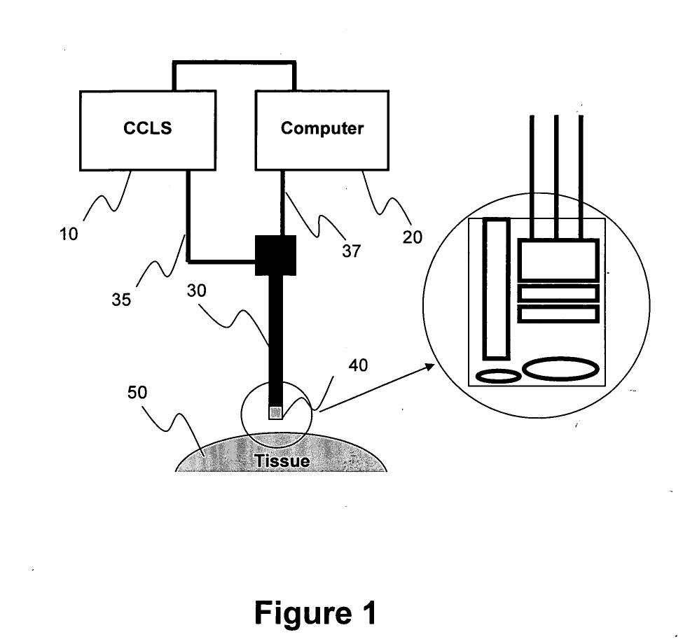

Turning to the figures, FIG. 1 schematically depicts a color endoscopy system 2. Computer controlled light source (CCLS) 10 is controlled by endoscopy system computerized controller 20, which is disposed at a proximal end of the light guide of endoscope 30. CCLS 30 emits a light beam that is directed into the illumination light guide 35 of endoscope 30. The light is conducted through the endoscope via the illumination light guide 35 to the distal tip 40 of the endoscope where it exits the endoscope and illuminates the tissue 50. A portion of the light emanating from tissue 50 is captured by the objective lens 43 located in endoscope tip 40 and is directed to form an image of the tissue on image sensor 45. Any suitable optical elements can be employed, such as lenses, mirrors, filters for the forming, mixing, imaging, collimating or other conditioning of the light as desired for objective lens 43. Thus, the light is passed by the objective either by transmitting the light or by reflecting the light or otherwise by acting upon the light. If desired, optical filters and other desired elements can also be provided in the primary image path, connected by mirrors, lenses or other optical components.

The image of the sample is transduced by image sensor 45 to create an electrical signal representative of the image. Image sensor 45 may be a charge coupled device (CCD), complementary metal oxide (CMOS) or charge injection device (CID) image sensor, or it may be another type of image sensor. The image sensor is a monochrome image sensor and is not equipped with a Bayer matrix type of wavelength selection optical filters disposed over the picture elements (pixels) of the image sensor, but has substantially the same wavelength sensitivity at all pixels of the image sensor. Image sensors that have the same wavelength response at all pixels are known as monochromatic image sensors or gray scale image sensors or often more simply as “black and white cameras”. It is an advantage to have a monochromatic image sensor and to rely on changing the spectral distribution of the illuminating light to provide improved endoscopes for color imaging of tissue.

While image sensor 45 is not equipped with wavelength selection filters for individual pixels it may have a simple optical filter 47 placed in front of the image sensor to select desired wavelengths or range of wavelengths that will impinge on all desired pixel elements, or to block undesired wavelengths or range of wavelengths. Examples of desired wavelengths could be wavelengths of the range visible to the human eye, wavelengths in the infrared or near infrared regions for thermal imaging, or other ranges desired for whatever the purpose might be. Undesired wavelengths might be wavelengths that interfere with sensor operation or reduce image contrast, because of chromatic aberration, or illumination wavelengths used for special purposes such as fluorescence excitation wavelengths that can be used for fluorescence imaging (or imaging of other emitted light) and but which may be desirable to block from impinging on the image sensor.

Image sensor 45 is operably connected via endoscope image output and image control cable 37 to the image capture system of endoscopy system controller 20. The image signal data from the image sensor 45 of endoscope 30 is transmitted to the system controller 20. The entire image or part of the image may be transmitted. The image pixel signals may be transmitted individually or they may be combined or otherwise processed prior to or after transmission. Transmission of the image signal may be effected by electrical signals traveling through conducting wires, optical signals traveling through optical fibers or other optical transmission methods or it may be transmitted by wireless communication devices such as radio waves or other types of wireless devices or networks, or otherwise as desired.

The system controller 20 captures the image signal and processes it and converts it to a digital image.

In another embodiment the system controller 20 captures the image signal and processes it as an analog signal.

The captured digital image is stored and associated with data that identifies the relative time the image was captured and the type of illumination provided by the CCLS when the image was captured. The system controller 20 can then process the images captured to present useful image information.

System controller 20 contains computer implemented programming that controls the spectral distribution and timing of the light output by the computer controlled light source 10.

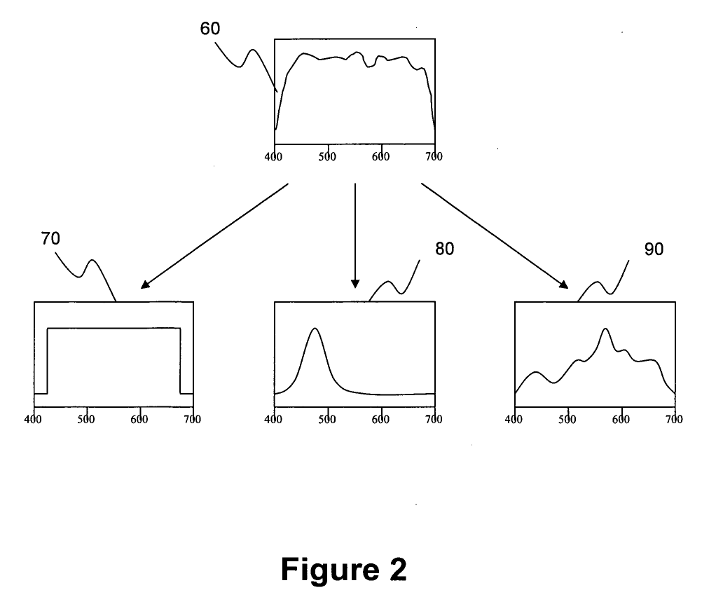

Turning to FIG. 2, the wavelength dependent distribution of energy, or spectrum of light emitted, from CCLS 10 can be represented graphically as intensity as a function of wavelength. An exemplary graph of the wavelength distribution of a white light source 60 is illustrative of the natural wavelength distribution of a xenon arc lamp as attenuated by the optical systems of the CCLS. The CCLS can control the relative energy distribution of the light emitted by the CCLS to reshape the spectrum emitted by the CCLS to provide an equalized energy spectrum 70 or a selected wavelength region for illumination 80, or an arbitrary spectral profile 90 that may enhance contrast for a feature of interest or provide other useful information when illuminating and imaging tissue.

An advantage of the controllability of the system is that providing an equalized energy distribution or a reference energy distribution allows correction of the output energy distribution for variations in lamp performance. This can be useful for maintaining consistency in imaging properties as lamps age and their spectral characteristics change.

A further advantage of the controllability of the illumination is that the dynamic range of the light available can be adjusted to provide the optimum level of illumination to complement the characteristics of the image sensor.

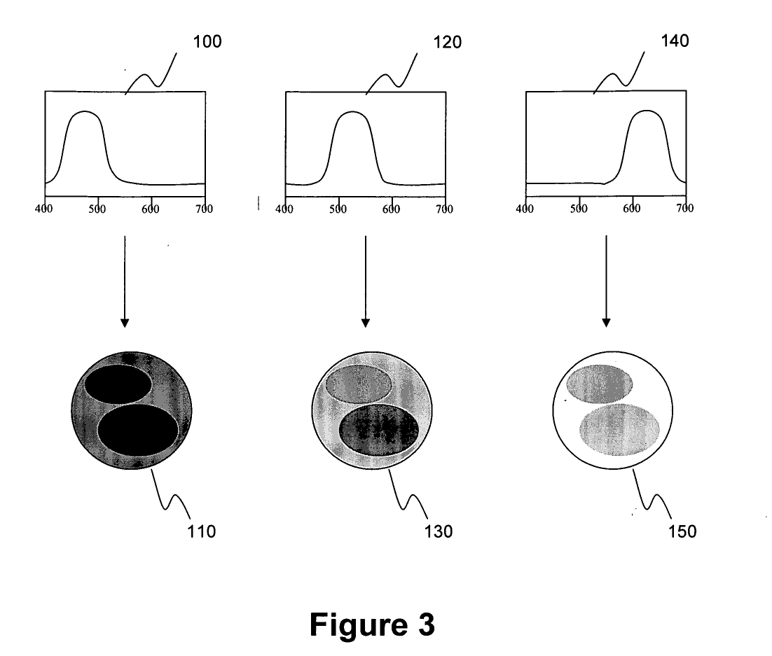

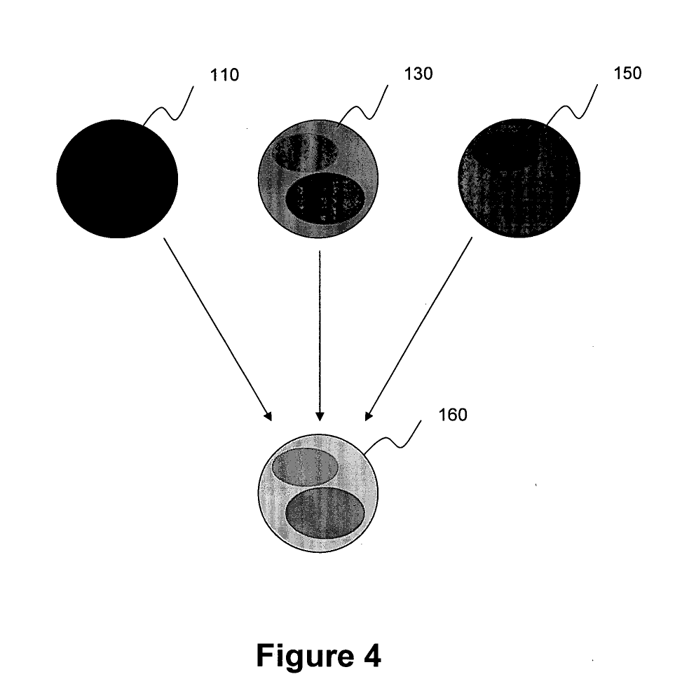

FIGS. 3 and 4 provide a schematic representation of capture of a sequence of images and their combination to form a color image of a tissue. In one phase, as shown in FIG. 3, CCLS 10 is operated under computer control to provide illumination by blue light, with an energy distribution represented by graph 100. The graph shows a selected wavelength distribution with an output ranging from approximately 400 nm to approximately 500 nm, which wavelengths correspond to those of blue light. This light illuminates the tissue 50 via endoscope 30 and an image is captured via image sensor 45 and relayed to the system controller where it is captured as first monochromatic image 110. CCLS 10 then changes its selected wavelength distribution to illuminate tissue 50 with the wavelength distribution corresponding to substantially green light as shown in graph 120, and system controller 20 captures second monochromatic image 130. CCLS 10 then changes its selected wavelength distribution to illuminate tissue 50 with the wavelength distribution corresponding to substantially red light as shown in graph 140 and system controller 20 captures third monochromatic image 150.

Monochromatic images 110, 130 and 150 are respectively assigned color channel values of blue, green and red by the digital image processing software that is part of the computer implemented programming of system controller 20. The image channels are then combined to form a color image 160 of tissue 50. By continuously repeating the capture and combination of the monochrome images into color images the system can provide a sequence of images that can be assembled into a video sequence that can show motion. The ability of the CCLS to change wavelengths quickly, the ability of the image sensor to capture images quickly, and the ability of the system controller to process images quickly provides an image capture system that can be operated to provide full motion video imaging that is perceptually comparable to video image sequences acquired by cameras that have simultaneous color image capture. If desired, the plurality of colors can, instead of rgb (red-blue-green), be cyan-yellow-magenta or any other combinations of colors, which combinations can comprise two, three, four or more colors.

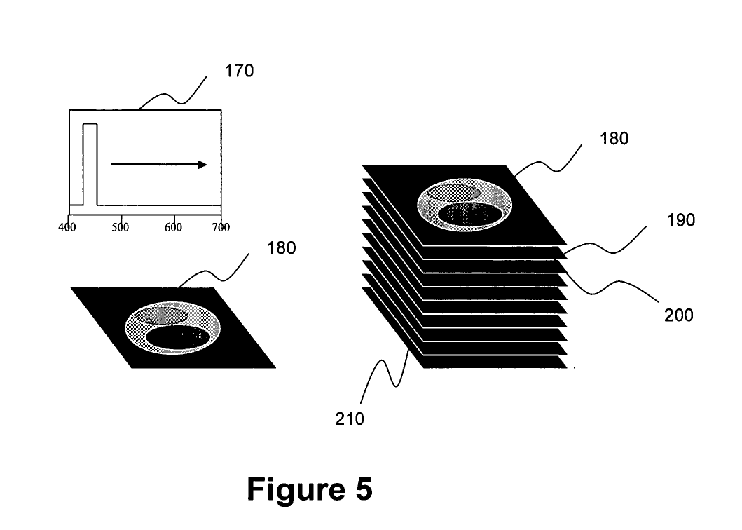

For example, as shown in FIG. 5, the CCLS can be configured to select a sequence of wavelength ranges that are much narrower in wavelength range that the red, green and blue ranges shown in FIG. 3. The CCLS can be programmatically controlled to sweep through a sequence of narrow wavelength ranges as illustrated in graph 170. Starting at the initial wavelength range, an image 180 is captured. As the CCLS sweeps through successive wavelength ranges, successive images such as image 190 and image 200 are captured. The wavelength range may be swept continuously, in a step-wise fashion, intermittently, randomly or otherwise as desired. The successive images are assembled into a “stack” of images often called an image cube 210. If a small number of images, for example three to ten images, are assembled then the image cube or stack is usually referred to as a multispectral image. If a large number of images, for example ten to hundreds of images, the image cube or stack is usually referred to as a hyperspectral image.

While hyperspectral images or multispectral images are often captured in sequences of wavelength ranges, this need not be the case exclusively. They can also be any combination of fluorescence images (or other emitted light images), reflectance images, polarized reflectance images of tissues, or otherwise as desired. They can further be combinations where the range of wavelengths of tissues is variable in bandwidth, or where the duration of exposure is variable.

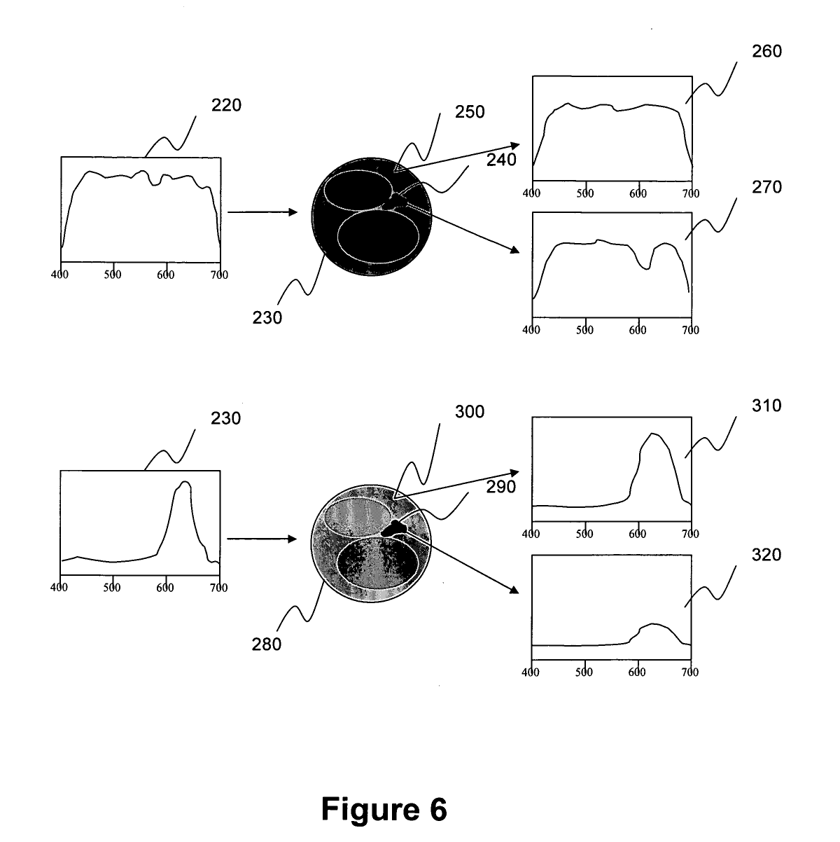

Turning to FIG. 6, it is possible to configure the computer controlled light source to select a wavelength distribution that enhances the contrast of a feature in the image. The CCLS can be programmatically controlled to produce an illumination spectral profile that interacts more strongly with the optical properties of a particular anatomical feature. By selecting a predetermined profile based on prior knowledge it is possible to select a range of profiles such that an operator can more effectively identify or understand the state of anatomical features in the tissue image. FIG. 6 schematically represents the process of modifying the spectral distribution of the illuminating light to enhance contrast of a tissue feature. The spectral distribution of an example of unconditioned or white light is represented in graph 220. White light 220 illuminates the tissue to produce monochromatic image 230. Image 230 has a feature of interest 240 in an area of normal tissue 250. The spectral reflectance characteristics of normal tissue 250 are shown in graph 260 and the spectral reflectance characteristics of feature of interest 240 are shown graphically in graph 270.

When a monochromatic image of a tissue is captured, all of the energy at all of the wavelengths emitted from the tissue, and that are within the wavelength response range of the detector and any filtering superimposed on the detector is collected for the area of the tissue corresponding to that pixel. The optical signal detected is thus the integrated intensity of the wavelengths emitted from that point in the tissue. It is proportional to the area under the tissue spectral curves of the tissue of interest for example normal tissue graph 260 and feature of interest graph 270. The ability to discriminate between these two signals is thus proportional to the difference or contrast between these two values.

To enhance contrast for a tissue of interest the illumination energy can be limited to wavelengths where the difference between the optical responses of the tissue is more significant. CCLS 10 can be controlled by system controller 20 to modify the spectral output of the CCLS to change the illumination of the tissue to a spectral profile that provides enhanced contrast, for example the spectral profile illustrated in graph 230 that illuminates the tissue and results in monochromatic image 280 being captured. The spectral response under this illumination of normal tissue area 300 is shown in graph 310. The spectral response under this illumination of area of interest 290 is shown in graph 320. The contrast ratio is proportional to the difference between the integrated intensity of the reflected illumination, represented by the area under the curve of the two spectral profiles. The difference between the integrated intensities for the contrast ratio is much greater for tissue illuminated by light with illumination spectrum 230 and thus the contrast ratio and the ability to identify the tissue of interest from the surrounding normal tissue is improved in the second image.

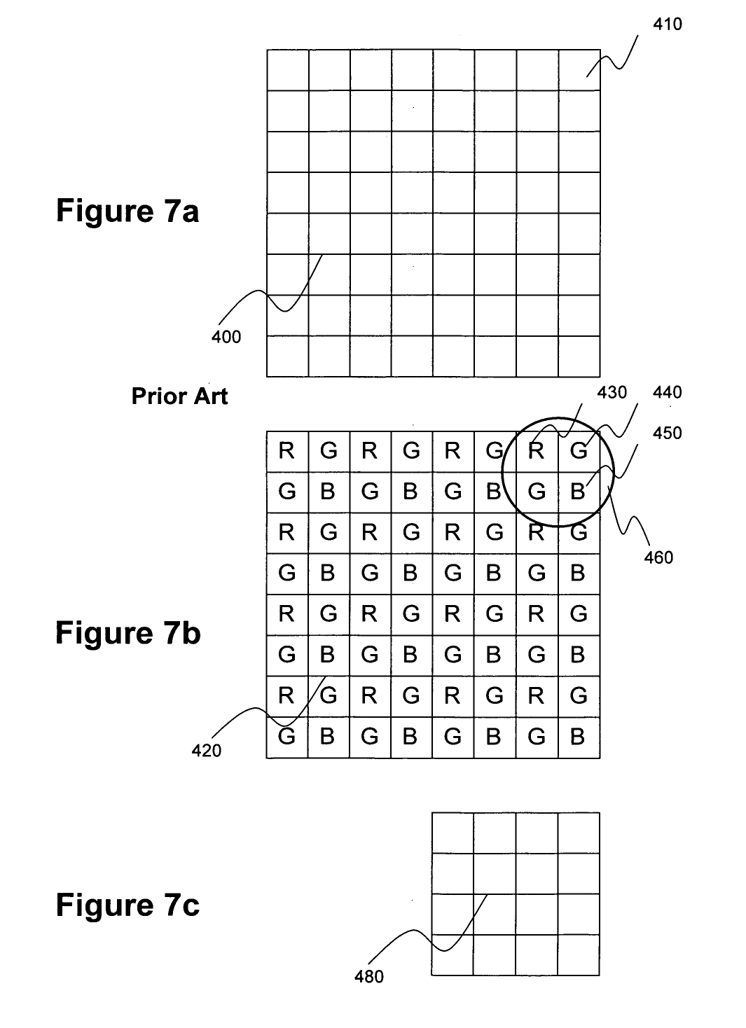

Turning to FIG. 7 and the construction of an endoscope using a monochrome image sensor, and a light source that can provide color images by varying the illumination light, a particular advantage of the systems, etc., herein is the ability to construct an endoscope that is much smaller than an endoscope that utilizes a color image sensor.

Generally, endoscopes currently available that integrate an image sensor in the tip of the endoscope use an image sensor with a matrix color filter superimposed over the image sensor. FIG. 7a illustrates schematically a typical arrangement of sensing elements into rectangular arrays to create a pixelated image sensor. The Figure shows a small portion of an image sensor 400 comprising an array of sensing elements 402, 404, 406, 408 and 410, each of which can be configured to provide a measurement value known as a picture element, or pixel for short, when each is configured to detect a different area of the sample image. In the present systems, this is achieved, for example, when each detects and transmits a grayscale reading. Each sensing element collects light from a portion of an image that is projected on it, creating measurement values comprising an intensity value and a location in space.

A well known way of producing a color image, as shown in FIG. 7b, is to create an array of optical filters that can be placed over individual sensing elements as illustrated schematically where a filter comprising red, green and blue filters has been arranged in a pattern that can be placed over image sensor 400. In this example, green filter 440 corresponds to the location of sensing element 410 and red filter 430 and blue filter 450 are positioned over adjacent sensing elements 440 and 450. In this example a second green filter is placed adjacent to green filter 440. This is one example of a pattern that could be used and is commonly referred to as a Bayer Pattern. Many patterns are known in the art, including patterns that have two red filters instead of two green filters, and patterns that include cyan, yellow, magenta and green filters for example.

When the image from a Bayer pattern type image sensor is read out the color of the filter over a sensing element location is known and thus the intensity value is assigned to the appropriate color channel. A group of four adjacent pixels thus forms a “color” pixel 460 for a particular location. To create a color image using a Bayer pattern on an image sensor means that four times as many camera pixels are required to create the same number of image pixels as a monochrome camera. A monochrome sensor can acquire an image of comparable resolution to a color sensor yet require only 25% of the active area. In other words, as opposed to the sensor in FIG. 7a where a single sensing element provides for a pixel, it takes four sensing elements to provide a single pixel in FIG. 7b.

FIG. 7c illustrates the relative size of a monochrome sensor 480 with the same pixel resolution of the color sensor 420 of FIG. 7b. It is well known that as image sensors are made smaller and smaller certain practical limits are reached. It is difficult to fabricate image sensors with pixels smaller that 6-8 microns in size. For an image sensor at the tip of an endoscope, the smaller the sensor the smaller the endoscope can be. A monochrome image sensor with a commonly favored image resolution such as 512×512 pixels would require at least an image sensor with a dimension of 512×6 microns which when calculated yields 3077 microns or just over 3 mm×3 mm. If this chip had a color filter matrix on it then the resolution would be halved to an image resolution of 256×256 pixels. Conversely, the systems, etc., herein provide for significantly better resolution than traditional Bayer Pattern-type color sensors when the sensor size is approximately the same; sensors of intermediate size provide better resolution and smaller size: as the size increases the total number of pixels increases.

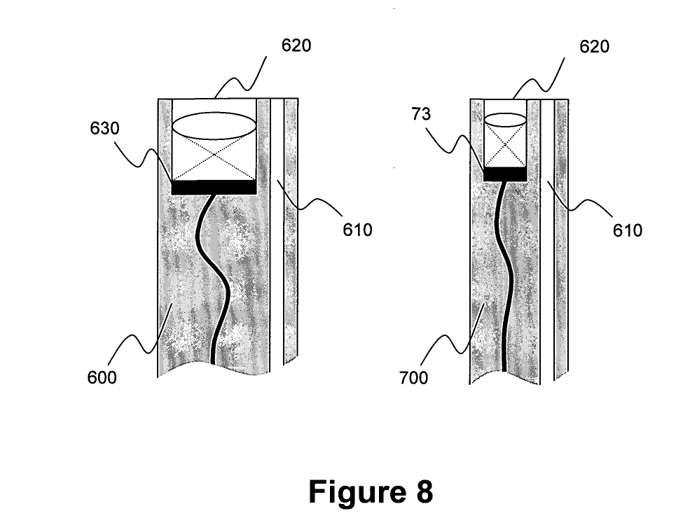

FIG. 8 shows a schematic representation of the effect on endoscope size of being configured to use a smaller image sensor. Image sensor 730 is 25% of the size of image sensor 630. The associated imaging objective is also smaller in size. This allows the overall size of endoscope body 600 to be reduced to the size of endoscope body 700. Thus, in some embodiments the endoscopes herein comprise color video-rate endoscopes having a diameter at the distal end of less than about 4 mm for a 512×512 pixel sensor having full intensity sensitivity, or less than about 3 mm for a 256×256 pixel sensor having full intensity sensitivity.

Thus an advantage of using a monochrome sensor and creating color images by sequentially illuminating with colored light is that image sensors with higher resolution but the same size, or with the same resolution but smaller in size allow the construction of smaller and/or better performing endoscopes, for example smaller in size with comparable image resolution or similar in size with improved image resolution.

Another advantage of using monochrome sensors is that monochrome sensors are substantially less costly to manufacture and require less circuitry and wires to transfer the image. The use of smaller and less costly components allows the construction of endoscopes that are significantly less costly. In some cases the cost is so low that the endoscope can be disposed of after a single use.

Thus, in some aspects the color imaging endoscope systems comprise an endoscope body including a proximal end and a distal end, the body configured to position the distal end proximate to a target tissue, a tunable light source configured to emit illumination light from the distal end comprising a variable selected spectral output and a variable wavelength dependent intensity distribution, a substantially monochromatic sensor disposed at the distal end and configured to detect light emanating from the target tissue and transmit a signal representing an intensity of the light to a processor, and a controller operably connected to the light source, the monochromatic sensor and the processor The controller contains computer-implemented programming configured to coordinate the light source, sensor and processor such that the light source provides over time a plurality of different desired wavelength bands of illumination light each having a selected, substantially pure, which means substantially only what's desired, it can be a simple band or complex set of wavelengths if desired, variable distribution and intensity. The monochromatic sensor detects light intensity emanating from the target tissue to provide a detected light intensity for each of the desired wavelength bands, and the processor associates the detected light intensity for each of the bands with a selected color suitable for display on a display device.

The selected color can be substantially the same as the desired wavelength band for each of the desired wavelength bands. The tunable light source can comprise a source of light and a tunable filter that provides the desired, wavelength and intensity variable, light. For example, tunable filter can comprise a spectrum former and a pixelated SLM, or an AOTF, configured to pass, over time, substantially only the desired wavelength bands of illumination light each having a selected, substantially pure, variable distribution and intensity. The tunable filter is operably connected to the controller, which contains computer-implemented programming that controls an on/off pattern of pixels in the pixelated SLM and/or the transmission characteristics of the AOTF to pass substantially only the desired wavelength bands of illumination light.

The SLM can be a reflective SLM, and the reflective surface can be configured to provide first and second pixelated SLM regions disposed substantially side-by-side with a light blocking barrier therebetween, with at least one optical element located and configured to transmit light from the first pixelated SLM region to the second pixelated SLM region. Other multiple filter and/or multiple SLM configurations, typically in series and combinations are also possible, for example multiple SLMs side-by-side, or nearby but operatively related, for example in the same optical path (upstream or downstream).

The different desired wavelength bands of illumination light can be red, green and blue, or cyan, yellow and magenta. They can also comprise at least four different bands configured for a multispectral image cube, large number of different bands configured for a hyperspectral image cube, or a plurality of intermittent spectra to provide a complex image cube. The wavelength bands can include fluorescence excitation illumination and the system further can comprise at least one long pass filter configured to block the fluorescence excitation illumination. The illumination light consists essentially of visible light, ultraviolet (UV) light or infrared (IR) light, or it can comprise combinations thereof. The different desired wavelength bands can be implemented sequentially in a repeated pattern or non-sequentially. Other color combinations and configurations are also possible.

The sensor can be configured to substantially only sense images. The distal end can contain a 512×512 pixel sensor having full intensity sensitivity yet with only a diameter less than about 4 mm, or a 256×256 pixel sensor with a diameter less than about 3 mm. The distal portion of the endoscope can be detachable and disposable, and the endoscope, or at least the distal portion, can flexible or non-flexible.

The computer implemented programming can be configured to selectively also provide a spectral output and a wavelength dependent intensity distribution that substantially mimics a spectral output and a wavelength dependent intensity distribution of output energy for disease treatment, photodynamic therapy, disease diagnosis, or to enhance contrast for detection or discrimination of a desired object in the target tissue. The system can be configured to provide different intensities for the plurality of different desired wavelength bands of illumination light by varying the amount of time the different desired wavelength bands can be emitted from the endoscope, and/or by attenuating the amount of light emitted for the different desired wavelength bands.

The processor can be the controller, and the systems further can comprise the display device.

Methods herein include methods of making the devices and systems herein, as well as methods of using such devices and systems. Exemplary methods include obtaining a color image of a target tissue through an endoscope by providing and emitting illumination light from the distal end of the endoscope to the target tissue to provide illuminated target tissue, the illumination light consisting essentially of a first desired wavelength band of illumination light having a selected, substantially pure, spectral output and wavelength dependent intensity distribution, substantially monochromatically detecting an intensity of light emanating from the illuminated target tissue to provide a first detected light intensity, and associating the first detected light intensity and the first desired wavelength band with a first selected color suitable for display on a display device. The process is then repeated for a second wavelength band and second color, then for a third wavelength band and color, as desired. The first and second selected colors can be substantially the same as the desired wavelength band for each of the desired wavelength bands. The methods further can comprise detecting the intensity of light emanating from the illuminated target tissue via a substantially monochromatic sensor disposed at the distal end, and otherwise using and implementing the devices and systems herein.

In some aspects, the present invention includes light engines and methods related thereto as discussed herein comprising specific, tunable light sources, which can be digital or non-digital. As noted elsewhere herein, one aspect of these systems and methods relates to the ability of the engines to provide finely tuned, variable wavelength ranges that correspond to precisely desired wavelength patterns, such as, for example, noon in Sydney Australia on October 14^{th} under a cloudless sky, or medically useful light of precisely 442 nm. For example, such spectra are created by receiving a dispersed spectrum of light from a typically broad spectrum light source (narrower spectrum light sources can be used for certain embodiments if desired) such that desired wavelengths and wavelength intensities across the spectrum can be selected by the digital light processor to provide the desired intensity distributions of the wavelengths of light. The remaining light from the original light source(s) is then shunted off to a heat sink, light sink or otherwise disposed of (in some instances, the unused light can itself be used as an additional light source, for metering of the emanating light, etc.).

In the present invention, either or both the light shunted to the heat sink or the light delivered to the target, or other light as desired, is measured. If the light is/includes the light to the light sink, then the measurement can, if desired, include a comparison integration of the measured light with the spectral distribution from the light source to determine the light projected from the light engine. For example, the light from the light sink can be subtracted from the light from the light source to provide by implication the light sent to a target. The light source is then turned up or down, as appropriate, so that as much light as desired is provided to the target, while no more light than desired, and no more power than desired, is emanated from or used by the light source. In the past, it was often undesirable to reduce or increase the power input/output of a given light source because it would change the wavelength profile of the light source. In the present system and methods, this is not an issue because the altered wavelength output of the light source is detected and the digital light processor is modified to adapt thereto so that the light ultimately projected to the target continues to be the desired wavelength intensity distribution.

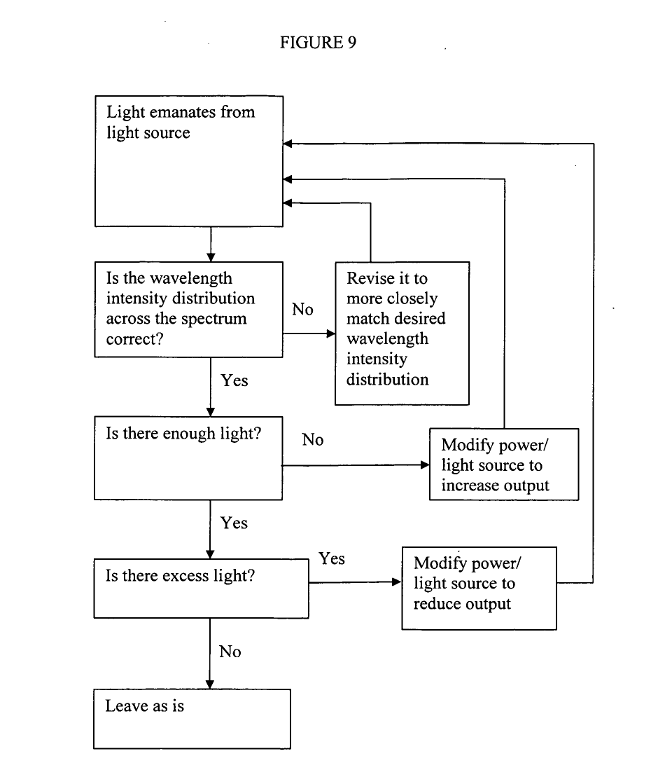

This aspect is depicted in a flow chart, FIG. 9, as follows: Is the wavelength intensity distribution across the spectrum correct? If yes, the proceed with the analysis; if no, then revise the wavelength intensity distribution across the spectrum as desired. Is the intensity target light distribution adequate? If no, then increase power output from light source and repeat. If yes, then proceed to next step. Is there excess light (for example being delivered to the light sink)? If yes, then decrease power to/from the light source. If no, then deem acceptable and leave as is. If power is increased or decreased: Re-check spectral distribution (e.g., of light emanated to target and/or of light from light power source) and if it is changed, reconfigure the digital light processor to adapt to the changed spectral input. If the light engine is changed, then reassess if light source can be turned up or down again. Repeat as necessary.

Some other advantages to the various embodiments herein is that the system is more power friendly, produces less heat, thereby possibly requiring fewer or less robust parts, and in addition should assist in increasing the longevity of various parts of the system due, for example, to the reduced heat generated and the reduced electricity transmitted and the reduced light transmitted. At the same time, this will provide the ability to use particular energy-favorable light sources that might not otherwise be able to be used due to fears over changed spectral distributions due to increased or decreased power output at the light source.

From the foregoing, it will be appreciated that, although specific embodiments have been discussed herein for purposes of illustration, various modifications may be made without deviating from the spirit and scope herein. Accordingly, the systems, methods, etc., herein include such modifications as well as all permutations and combinations of the subject matter set forth herein and is not limited except as by the appended claims.

1. A color imaging endoscope system comprising:
a) an endoscope body including a proximal end and a distal end, the body configured to position the distal end proximate to a target tissue;
b) a tunable light source configured to emit from the distal end an illumination light comprising a selectively variable selected spectral output and a selectively variable wavelength dependent intensity distribution;
c) a substantially monochromatic sensor disposed at the distal end and configured to detect light emanating from the target tissue and to transmit a signal representing an intensity of the light to a processor; and,
d) a controller operably connected to the light source, the monochromatic sensor and the processor, the controller containing computer-implemented programming configured to coordinate the light source, sensor and processor such that the light source provides over time a plurality of different desired wavelength bands of illumination light each having a selected, substantially pure, variable distribution and intensity, the monochromatic sensor detects light intensity emanating from the target tissue to provide a detected light intensity for each of the desired wavelength bands, and the processor associates the detected light intensity for each of the bands with a selected color suitable for display on a display devices
wherein the tunable light source comprises:
a) a source of light,
b) a tunable filter comprising:
a spectrum former able to provide a spectrum from a light beam traveling along a light path from the source of light,
a pixelated spatial light modulator (SLM) located downstream from and optically connected to the spectrum former, the pixelated SLM configured to pass, over time, substantially only the desired wavelength bands of illumination light each having a selected, substantially pure, variable distribution and intensity, the pixelated SLM operably connected to the controller, which contains computer-implemented programming that controls an on/off pattern of pixels in the pixelated SLM to pass substantially only the desired wavelength bands of illumination light.

2-9. (canceled)

10. The endoscope system of claim 1 wherein the different desired wavelength bands comprise at least four different wavelength bands.

11-13. (canceled)

14. The endoscope system of claim 1 wherein the substantially monochromatic sensor is configured to substantially only sense images.

15. The endoscope system of claim 1 wherein the distal end contains a pixel sensor having at least 512×512 pixels having full intensity sensitivity and has a diameter less than about 4 mm.

16. The endoscope system of claim 1 wherein the distal end contains a pixel sensor having at least 256×256 pixels having full intensity sensitivity and has a diameter less than about 3 mm.

17. The endoscope system of claim 1 wherein the endoscope is configured such that a distal portion of the endoscope is detachable and disposable.

18. The endoscope system of claim 1 wherein the body of the endoscope is non-flexible.

19-29. (canceled)

30. The endoscope system of claim 1 wherein the system further comprises the display device.

31. (canceled)

32. The endoscope system of claim 1 wherein the system is configured to provide different intensities for the plurality of different desired wavelength bands of illumination light by attenuating the amount of light emitted for the different desired wavelength bands.

33-68. (canceled)

69. A color imaging system comprising:
a) a tunable light source configured to emit to a target an illumination light comprising a selectively variable selected spectral output and a selectively variable wavelength dependent intensity distribution;
b) a substantially monochromatic sensor configured to detect light emanating from the target and to transmit a signal representing an intensity of the light to a processor; and,
c) a controller operably connected to the light source, the monochromatic sensor and the processor, the controller containing computer-implemented programming configured to coordinate the light source, sensor and processor such that the light source provides over time a plurality of different desired wavelength bands of illumination light each having a selected, substantially pure, variable distribution and intensity, the monochromatic sensor detects light intensity emanating from the target to provide a detected light intensity for each of the desired wavelength bands, and the processor associates the detected light intensity for each of the bands with a selected color suitable for display on a display device,
wherein the tunable light source comprises:
a) a source of light,
b) a tunable filter comprising:
a spectrum former able to provide a spectrum from a light beam traveling along a light path from the source of light,
a pixelated spatial light modulator (SLM) located downstream from and optically connected to the spectrum former the pixelated SLM configured to pass, over time, substantial only the desired wavelength bands of illumination light each having a selected, substantially pure, variable distribution and intensity, the pixelated SLM operably connected to the controller, which contains computer-implemented programming that controls an on/off pattern of pixels in the pixelated SLM to pass substantially only the desired wavelength bands of illumination light.

70-131. (canceled)
